(19) United States
(12) Patent Application Publication
(10) Pub. No.: US 2011/0136197 A1
(43) Pub. Date: Jun. 9, 2011

(54) Expression of Catalase in Trichoderma

(76) Inventors: Timothy C Dodge, Sunnyvale, CA (US); Katherine M. Hoffmann, Palo Alto, CA (US); Andrei Miasnikov, Mountain View, CA (US); Michael Ward, San Francisco, CA (US)

(21) Appl. No.: 12/920,688

(22) PCT Filed: Mar. 5, 2009
(86) PCT No.: PCT/US09/36146

§ 371 Date: Dec. 14, 2010

Related U.S. Application Data
(60) Provisional application No. 61/034,788, filed on Mar. 7, 2008.

(51) Int. Cl. C12P 3/00; C12N 9/08

(52) U.S. Cl. 435/168; 435/192

(57) ABSTRACT

The invention provides methods for expression of a catalase enzyme in a Trichoderma host cell. In one embodiment, the catR gene from Aspergillus niger is expressed in Trichoderma reesei, resulting in improved yields of catalase enzyme in comparison with expression of catR in A. niger.

### CROSS-REFERENCE TO RELATED APPLICATIONS

This application claims the benefit of U.S. Provisional Application No. 61/034,788, filed on Mar. 7, 2008, which is incorporated by reference herein in its entirety.

### FIELD OF THE INVENTION

The invention relates to expression of a catalase enzyme in a Trichoderma host cell, particularly expression of the catR gene from Aspergillus niger in Trichoderma reesei.

### BACKGROUND

Catalases [hydrogen peroxide:hydrogen peroxide oxidoreductases (EC 1.11.1.6)] are enzymes that catalyze the conversion of hydrogen peroxide (H₂O₂) to oxygen (O₂) and water (H₂O):

2H₂O₂→2H₂O+O₂

Catalases have been purified from a number of animal tissues, plants, and microorganisms (Chance and Maehly (1955) Methods in Enzymology 2:764-791; Jones and Wilson (1978) in H. Sigel (ed.), Metal Ions in Biological Systems, Vol. 7, Marcel Dekker Inc., New York). Most catalase enzymes that have been characterized contain four polypeptide subunits, each having a molecular weight of 50,000 to 60,000, and one protohemin prosthetic group per subunit (Wasserman and Hultin (1981) Arch. Biochem. Biophys. 212:385-392; Hartig and Ruis (1986) Eur. J. Biochem. 160:487-490). Catalase from bovine liver has been the most extensively studied enzyme (Schonbaum and Chance (1976) in The Enzymes, P. D. Boyer, ed., 3^{rd} edition, Vol. 13, pp. 363-408, Academic Press, New York). The complete amino acid sequence and three dimensional structure of bovine liver catalase are known (Schroeder et al. (1983) Arch. Biochem. Biophys. 214:397-412; Murphy et al. (1981) J. Mol. Biol. 152:465-499).

Although less well-studied from a biochemical and biophysical standpoint, catalases from filamentous fungi have characteristics that distinguish them and provide advantages over their mammalian counterparts. While similar in subunit number and heme content, fungal catalases are substantially larger molecules than those from other organisms, having subunit molecular weights ranging from 80,000 to 97,000. (Vainshtein et al. (1986) J. Mol. Biol. 188:63-72; Jacob and Orme-Johnson (1979) Biochem. 18:2967-2975; Jones et al. (1987) Biochim. Biophys. Acta 913:395-398) More importantly, catalases from fungal species such as Aspergillus niger are more stable than beef liver catalase to proteolysis and to inactivation by glutaraldehyde or SDS, and have lower affinity for catalase inhibitors such as cyanide, azide, and fluoride (Wasserman and Hultin, supra). In addition, A. niger catalase is significantly more stable than beef liver catalase when subjected to extremes of pH, hydrogen peroxide concentration, and temperature (Scott and Hammer (1960) Enzymologia 22:229-237). Although fungal catalase offers stability advantages, the corresponding mammalian enzymes such as beef liver catalase appear to have higher catalytic activity (Gruft et al. (1978) Can. J. Biochem. 56(9):916-919; Kikuchi-Torii et al. (1982) J. Biochem. (Tokyo) 92(5):1449-1456). However, since enzyme stability is an important factor in the biotechnological utilization of enzymes, there has been considerable interest in the use of fungal catalases, especially for applications involving neutralization of high concentrations of hydrogen peroxide. It has been observed that the rate of deactivation in H₂O₂ was at least an order of magnitude lower for A. niger catalase than for beef liver catalase (Vasudevan and Weiland (1990) Biotechnol. Bioeng. 36:783-789). The differences in stability between the two enzymes may be attributable to differences in structural characteristics and composition of the proteins (Vasudevan and Weiland, supra).

Traditionally, beef liver catalase has been the preferred enzyme for diagnostic purposes and for pharmaceutical-related applications (e.g., contact lens cleaning, disinfection, H₂O₂ neutralization). However, concern over bovine spongiform encephalopathy in European cattle and fear that the disease may be contracted by humans (Dealler and Lacey (1991) Neutr. Health (Bicester) 7:117-134; Dealler and Lacey (1990) Food Microbiol. 7:253-280) have aroused interest in alternative catalase sources.

Catalase preparations from A. niger are sold commercially for diagnostic enzyme kits, for the enzymatic production of sodium gluconate from glucose, for the neutralization of H₂O₂ waste, and for the removal of H₂O₂ and/or generation of O₂ in food and beverages. Two catalase genes have been isolated from A. niger. The A. niger catA gene encodes a catalase enzyme that is induced primarily during growth on fatty acids and is thought to be located in peroxisomes. A second gene, designated catR, encodes a soluble cytoplasmic enzyme, which represents the major activity in commercial A. niger catalase preparations.

Recombinant expression of the A. niger catR gene, to which promoter and terminator elements of the A. niger glucoamylase (glaA) gene are functionally attached, has been previously described in an A. niger strain engineered to eliminate glucose oxidase (goxA) expression (U.S. Pat. No. 5,360,901). However, yields of the enzyme are not optimal because a portion of the expressed catalase enzyme is sequestered in the cell walls of the A. niger host cells, decreasing enzyme yield and recovery. Therefore, there is a need for an improved host expression system for final catalase that will provide a greater yield of secreted soluble enzyme.

### BRIEF SUMMARY OF THE INVENTION

In one aspect, the invention provides a method for producing a polypeptide capable of catalyzing enzymatic conversion of hydrogen peroxide to oxygen and water, including expressing the polypeptide from a polynucleotide that encodes the polypeptide in a Trichoderma host cell. In one embodiment, the polypeptide is a catalase enzyme from Aspergillus niger (A. niger). In one embodiment, the polynucleotide contains the A. niger catR gene. In one embodiment, the polynucleotide encodes a polypeptide containing the amino acid sequence set forth in SEQ ID NO:1. In one embodiment, the Trichoderma host cell is a Trichoderma reesei (T. reesei) cell. In some embodiments, expression of the polypeptide in T. reesei is at least about any of 20, 30, 40, 50, 60, 70, 80, 90, or 100% greater than expression of the same polypeptide in A. niger. In some embodiment, the amount of the polypeptide secreted into the growth medium from the T. reesei host cell is at least about any of 20, 30, 40, 50, 60, 70, 80, 90, or 100% higher than the amount of the polypeptide secreted into the growth medium for an A. niger host cell.

In one embodiment, the method includes (a) transforming a Trichoderma host cell with an expression vector comprising a polynucleotide that encodes said polypeptide; (b) growing said Trichoderma host cell in a growth medium under conditions suitable for expression of said polypeptide; and (c) isolating said polypeptide from said growth medium.

In another aspect, the invention provides a Trichoderma host cell containing an expression vector that contains a polynucleotide that encodes a polypeptide that is capable of catalyzing enzymatic conversion of hydrogen peroxide to oxygen and water. In one embodiment, the polypeptide is a catalase enzyme from A. niger. In one embodiment, the polynucleotide contains the A. niger catR gene. In another embodiment, the polynucleotide encodes a polypeptide containing the amino acid sequence set forth in SEQ ID NO:1. In one embodiment, the host cell is a T. reesei cell. In some embodiments, the Trichoderma host cell comprises a deletion of the endo-T gene. In one embodiment, the host cell is a T. reesei cell with a deletion of the endo-T gene.

In another aspect, the invention provides a polypeptide that is capable of catalyzing enzymatic conversion of hydrogen peroxide to oxygen and water, wherein the polypeptide is expressed in a Trichoderma host cell as described herein.

In another aspect, the invention provides a method for converting hydrogen peroxide to oxygen and water, including contacting the hydrogen peroxide with a polypeptide that is capable of catalyzing enzymatic conversion of hydrogen peroxide to oxygen and water, wherein the polypeptide is expressed in a Trichoderma host cell as described herein.

In another aspect, the invention provides a composition for conversion of hydrogen peroxide to oxygen and water, including a polypeptide that is capable of catalyzing enzymatic conversion of hydrogen peroxide to oxygen and water, wherein the polypeptide is expressed in a Trichoderma host cell as described herein.

In another aspect, the invention provides a kit including a polypeptide that is capable of catalyzing enzymatic conversion of hydrogen peroxide to oxygen and water, wherein the polypeptide is expressed in a Trichoderma host cell as described herein.

### BRIEF DESCRIPTION OF THE DRAWINGS

FIG. 1 depicts the amino acid sequence of the A. niger catR catalase enzyme (SEQ ID NO:1). The signal sequence that directs secretion is indicated in italics.

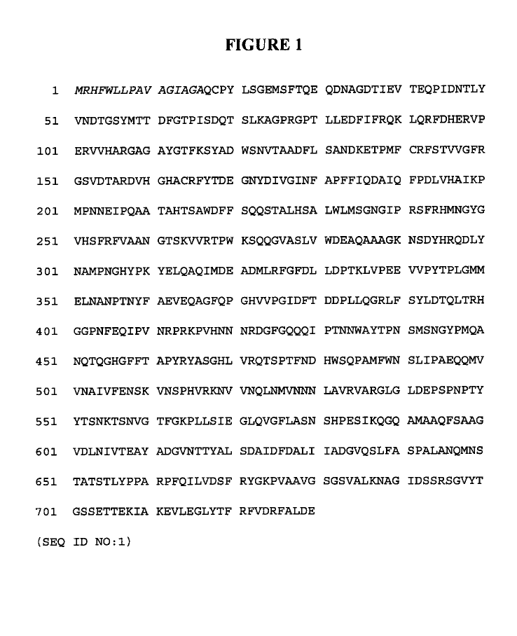

FIG. 2 depicts the nucleotide sequence that encodes the A. niger catR catalase enzyme, including introns (SEQ ID NO:2).

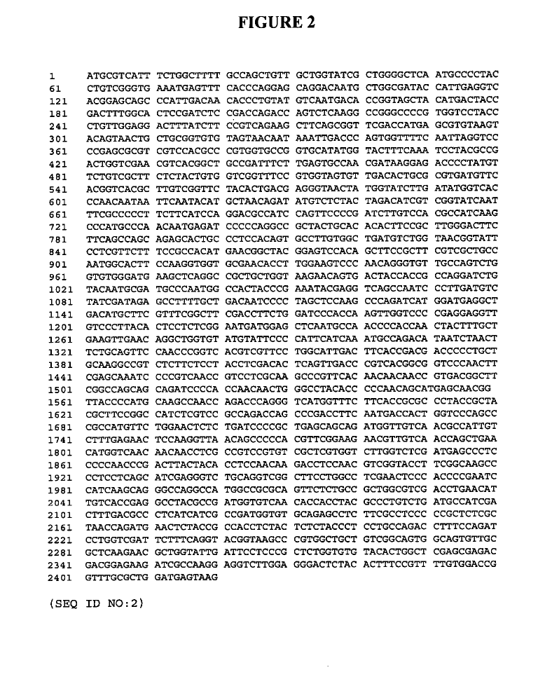

FIG. 3 depicts the structure of the pTrex3gM expression vector.

FIG. 4 depicts the structure of the pTrex3gM(CATE) expression vector.

FIG. 5 shows the effect of pH on melting point of catalase enzyme expressed in A. niger or T. reesei.

FIG. 6 shows the effect of H₂O₂ on melting point of catalase enzyme expressed in A. niger or T. reesei.

FIG. 7 is an SDS-PAGE gel as described in Example 6.

FIG. 8 shows activity of catalase samples as described in Example 7.

### DETAILED DESCRIPTION

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### DEFINITIONS

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide” and “nucleic acid” and “oligonucleotide” are used herein interchangeably. Polynucleotides of the invention may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, “polypeptide” refers to any composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “vector” refers to a polynucleotide sequence designed to introduce nucleic acids into one or more cell types. Vectors include cloning vectors, expression vectors, shuttle vectors, plasmids, phage particles, cassettes and the like.

As used herein, the term “expression” refers to the process by which a polypeptide is produced based on the nucleic acid sequence of a gene. The process includes both transcription and translation.

As used herein, “expression vector” refers to a DNA construct containing a DNA coding sequence (e.g., gene sequence) that is operably linked to one or more suitable control sequence(s) capable of effecting expression of the coding sequence in a host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. The plasmid is the most commonly used form of expression vector. However, the invention is intended to include such other forms of expression vectors that serve equivalent functions and which are, or become, known in the art.

A “promoter” refers to a regulatory sequence that is involved in binding RNA polymerase to initiate transcription of a gene. The promoter may be an inducible promoter or a constitutive promoter. A non-limiting example of an inducible promoter which may be used in the invention is Trichoderma reesei cbh1, which is an inducible promoter.

The term “operably linked” refers to juxtaposition wherein the elements are in an arrangement allowing them to be functionally related. For example, a promoter is operably linked to a coding sequence if it controls the transcription of the coding sequence.

“Under transcriptional control” is a term well understood in the art that indicates that transcription of a polynucleotide sequence depends on its being operably linked to an element which contributes to the initiation of, or promotes transcription.

“Under translational control” is a term well understood in the art that indicates a regulatory process which occurs after mRNA has been formed.

A “gene” refers to a DNA segment that is involved in producing a polypeptide and includes regions preceding and following the coding regions as well as intervening sequences (introns) between individual coding segments (exons).

As used herein, the term “host cell” refers to a cell or cell line into which a recombinant expression vector for production of a polypeptide may be transfected for expression of the polypeptide. Host cells include progeny of a single host cell, and the progeny may not necessarily be completely identical (in morphology or in total genomic DNA complement) to the original parent cell due to natural, accidental, or deliberate mutation. A host cell includes cells transfected or transformed in vivo with an expression vector. “Host cell” refers to both cells and protoplasts created from the cells of a filamentous fungal strain and particularly a Trichoderma sp. strain.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “signal sequence” (also termed “presequence,” “signal peptide,” “leader sequence,” or “leader peptide”) refers to a sequence of amino acids bound to the N-terminal portion of a protein which facilitates the secretion of the mature form of the protein from the cell. The signal sequence targets the polypeptide to the secretory pathway and is cleaved from the nascent polypeptide once it is translocated in the endoplasmic reticulum membrane. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “derived from” encompasses the terms “originated from,” “obtained from,” “obtainable from,” “isolated from,” and “created from.”

“Filamentous fungi” refers to all filamentous forms of the subdivision Eumycotina (See, Alexopoulos, C. J. (1962), Introductory Mycology, Wiley, New York). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligatorily aerobic. In the present invention, the filamentous fungal parent cell may be a cell of a species of Trichoderma, e.g., Trichoderma reesei (previously classified as T. longibrachiatum and currently also known as Hypocrea jecorina), Trichoderma viride, Trichoderma koningii, or Trichoderma harzianum.

As used herein, the term “Trichoderma” or “Trichoderma sp.” refers to any fungal genus previously or currently classified as Trichoderma.

The term “culturing” refers to growing a population of microbial cells under suitable conditions for growth, in a liquid or solid medium.

The term “heterologous” in reference to a polynucleotide or protein refers to a polynucleotide or protein that does not naturally occur in a host cell. In some embodiments, the protein is a commercially important industrial protein. It is intended that the term encompass proteins that are encoded by naturally occurring genes, mutated genes, and/or synthetic genes. The term “homologous” in reference to a polynucleotide or protein refers to a polynucleotide or protein that occurs naturally in the host cell.

The term “introduced” in the context of inserting a nucleic acid sequence into a cell includes “transfection,” “transformation,” or “transduction” and refers to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed.

As used herein, the terms “transformed,” “stably transformed,” and “transgenic” refer to a cell that has a non-native (e.g., heterologous) nucleic acid sequence integrated into its genome or as an episomal plasmid that is maintained through multiple generations.

The terms “recovered,” “isolated,” “purified,” and “separated” as used herein refer to a material (e.g., a protein, nucleic acid, or cell) that is removed from at least one component with which it is naturally associated. For example, these terms may refer to a material which is substantially or essentially free from components which normally accompany it as found in its native state, such as, for example, an intact biological system.

“Catalase” as used herein refers to an enzyme (i.e., a polypeptide having catalytic activity) that catalyzes the decomposition of hydrogen peroxide to hydrogen and oxygen.

“ATCC” refers to American Type Culture Collection located at Manassas, Va. 20108 (www.atcc.org).

“NRRL” refers to the Agricultural Research Service Culture Collection, National Center for Agricultural Utilization Research (and previously known as USDA Northern Regional Research Laboratory), Peoria, Ill.

“A,” “an” and “the” include plural references unless the context clearly dictates otherwise.

### Trichoderma Host Cells

The invention provides host cells that can express a heterologous polynucleotide encoding a catalase enzyme. The host cell is a filamentous fungal cell of a Trichoderma species, for example, Trichoderma reesei, Trichoderma viride, Trichoderma koningii, or Trichoderma harzianum. In some embodiments, the host cell is a Trichoderma reesei strain. Strains of T. reesei are known; nonlimiting examples include ATCC No. 13631, ATCC No. 26921, ATCC No. 56764, ATCC No. 56765, ATCC No. 56767, and NRRL No. 15709. In some embodiments, the host cell is derived from the RL-P37 T. reesei strain (described in Sheir-Neiss et al. (1984) Appl. Microbiol. Biotechnology 20:46-53. In one embodiment, the host cell is the Morph 1.1 (pyr+) T. reesei strain, a spontaneous pyr4 revertant of the quad-deleted RL-P37 Trichoderma reesei strain (described in PCT Application No. WO 05/001036).

The host strain may have been previously manipulated through genetic engineering. In some embodiments, various native genes of the fungal host cell have been inactivated. The genes include, for example, genes encoding cellulolytic enzymes, such as endoglucanases (EG) and exocellobiohydrolases (CBH) (e.g., cbh1, cbh2, egl1, and egl3). U.S. Pat. No. 5,650,322 discloses derivative strains of RL-P37 having deletions in the cbh1 gene and the cbh2 gene. In some embodiments, the Trichoderma host cell contains a deletion of the endo-T gene, to reduce or prevent deglosylation of the expressed catalase enzyme. In one embodiment, the host cell is Trichoderma reesei with a deletion of the endo-T gene.

An expression vector encoding a polypeptide of the invention can be transfected into a host cell using standard techniques. “Transfection” or “transformation” refers to the insertion of an exogenous polynucleotide into a host cell. The exogenous polynucleotide may be maintained as a non-integrated vector, for example, a plasmid, or alternatively, may be integrated into the host cell genome. The term “transfecting” or “transfection” is intended to encompass all conventional techniques for introducing nucleic acid into host cells. Examples of transfection techniques include, but are not limited to, calcium phosphate precipitation, DEAE-dextran-mediated transfection, lipofection, electroporation, and microinjection. Suitable methods for transfecting host cells can be found in Sambrook et al. (1989) Molecular Cloning: A Laboratory Manual, 2nd Edition, Cold Spring Harbor Laboratory press, and other laboratory textbooks. Nucleic acid can also be transferred into cells via a delivery mechanism suitable for introduction of nucleic acid into cells in vivo, such as via a retroviral vector (see e.g., Ferry et al. (1991) Proc. Natl. Acad. Sci., USA, 88: 8377-8381; and Kay et al. (1992) Human Gene Therapy 3: 641-647), an adenoviral vector (see, e.g., Rosenfeld (1992) Cell 68: 143-155; and Herz and Gerard (1993) Proc. Natl. Acad. Sci., USA, 90:2812-2816), receptor-mediated DNA uptake (see e.g., Wu, and Wu (1988) J. Biol. Chem. 263: 14621; Wilson et al. (1992) J. Biol. Chem. 267: 963-967; and U.S. Pat. No. 5,166,320), direct injection of DNA (see, e.g., Acsadi et al. (1991) Nature 332: 815-818; and Wolff et al. (1990) Science 247:1465-1468) or particle bombardment (biolistics) (see e.g., Cheng et al. (1993) Proc. Natl. Acad. Sci., USA, 90:4455-4459; and Zelenin et al. (1993) FEBS Letts. 315: 29-32).

Certain vectors integrate into host cells at a low frequency. In order to identify these integrants, in some embodiments a gene that contains a selectable marker (e.g., drug resistance) is introduced into host cells along with the nucleic acid of interest. Examples of selectable markers include those which confer resistance to certain drugs, such as G418 and hygromycin. Selectable markers can be introduced on a separate vector from the nucleic acid of interest or on the same vector. Transfected host cells can then be identified by selecting for cells using the selectable marker. For example, if the selectable marker encodes a gene conferring neomycin resistance, host cells which have taken up nucleic acid can be identified by their growth in the presence of G418. Cells that have incorporated the selectable marker gene will survive, while the other cells die.

Once expressed, a polypeptide of the invention can be purified according to standard procedures of the art, including, but not limited to affinity purification, ammonium sulfate precipitation, ion exchange chromatography, or gel electrophoresis (see generally, R. Scopes (1982) Protein Purification, Springer-Verlag, N.Y.; Deutscher (1990) Methods in Enzymology Vol. 182: Guide to Protein Purification, Academic Press, Inc. N.Y.).

### Catalase Enzymes

In the context of this invention, a catalase is an enzyme that catalyzes the conversion of hydrogen peroxide to oxygen and water. Catalase enzymes expressed in Trichoderma host cells as described herein are heterologous to the host species, i.e., they are derived from a different species than the Trichoderma species in which they are expressed.

In one embodiment, the catalase is the catR catalase of A. niger. In some embodiments, the catalase comprises, consists of, or consists essentially of the amino acid sequence set forth in SEQ ID NO:1. In some embodiments, the catalase comprises, consists of, or consists essentially of an amino acid sequence with any of about 70, 75, 80, 85, 90, 95, 97, 98, 99, or 99.5% identity with the sequence set forth in SEQ ID NO:1, wherein the enzyme is capable of catalyzing the conversion of hydrogen peroxide to oxygen and water.

In some embodiments the catalase is a mature processed sequence derived from the amino acid sequence set forth in SEQ ID NO:1. In some embodiments, the mature processed sequence derived from the amino acid sequence set forth in SEQ ID NO:1 contains a deletion of 1 to about 25 amino acid residues from the N-terminus of SEQ ID NO:1, such as a deletion of 1, 2, 3, 4, 5, 6, 7, 8, 9, 10, 11, 12, 13, 14, 15, 16, 17, 18, 19, 20, 21, 22, 23, 24, or 25 N-terminal amino acids.

In one embodiment, the catalase is encoded by the catR gene of A. niger. In some embodiments, the catalase is encoded by a polynucleotide that comprises, consists of, or consists essentially of the polynucleotide sequence set forth in SEQ ID NO:2. In some embodiments, the catalase is encoded by a polynucleotide sequence with any of about 70, 75, 80, 85, 90, 95, 97, 98, 99, or 99.5% identity with the polynucleotide sequence set forth in SEQ ID NO:2, wherein the enzyme encoded by the polynucleotide is capable of catalyzing the conversion of hydrogen peroxide to oxygen and water.

### Vectors

In accordance with the invention, a vector comprising a polynucleotide sequence that encodes a catalase enzyme is introduced into a Trichoderma host cell. Typically, a polypeptide comprising catalase activity is expressed from an expression vector comprising a polynucleotide encoding the polypeptide and comprising regulatory sequence(s) operably linked to the catalase coding sequence.

The vector may be any vector that can be introduced into and replicated in a Trichoderma cell. Examples of suitable vectors may be found, for example, in Sambrook et al. (1989), supra, Ausubel (1994), supra, van den Hondel et al. (1991) in Bennet and Lasure (Eds.), More Gene Manipulations in Fungi, Academic Press, San Diego, pp. 396-428, and U.S. Pat. No. 5,874,276. Examples of useful vectors include, but are not limited to, pFB6, pBR322, PUC18, pUC100, and pENTR/D.

Typically, a polynucleotide encoding a catalase enzyme is operably linked to a suitable promoter that exhibits transcriptional activity in the Trichoderma host cell. The promoter may be derived from a gene encoding a protein that is either homologous or heterologous to the host cell. Suitable nonlimiting examples of promoters include cbh1, cbh2, egl1, and egl2. In some embodiments, the promoter is native to the host cell. For example, when T. reesei is the host cell, the promoter may be a native T. reesei promoter. In one embodiment, the promoter is T. reesei cbh1, which is an inducible promoter and has been deposited in GenBank under Accession No. D86235. An “inducible promoter” is a promoter that is active under environmental or developmental regulatory conditions. In another embodiment, the promoter is heterologous to the host cell.

In some embodiments, the catalase coding sequence is operably linked to a polynucleotide sequence encoding a signal sequence. In some embodiments, the signal sequence is one that is naturally associated with the catalase gene to be expressed. In some embodiments, the signal sequence comprises, consists of, or consists essentially of the amino acid sequence depicted in residues 1 to 16 of SEQ ID NO:1. In some embodiments, the signal sequence has least about 80, 85, 90%, 95%, 97%, 98%, or 99% sequence identity with the signal sequence depicted in amino acid residues 1 to 16 of SEQ ID NO:1. In some embodiments, the signal sequence is the T. reesei cbh1 or nsp24 signal sequence. In one embodiment, the signal sequence is the amino acid sequence encoded by the cbh1 nucleic acid sequence ATGTATCGGAAGTTGGCCGTCATCTCGGCCTTCTTGGCCACAGCTCGTGCT (SEQ ID NO:3). In one embodiment, the signal sequence is the amino acid sequence encoded by the nsp24 nucleic acid sequence ATGCAGACCTTTGGAGCTTTTCTCGTTTCCTTCCTCGCCGCCAGCGGCCTGGCCGCGGCC (SEQ ID NO:4). In some embodiments, a vector to be introduced into a Trichoderma host cell comprises a signal sequence and a promoter sequence derived from the same source. In some embodiments, the signal sequence and the promoter sequence are derived from different sources.

In some embodiments, the vector also includes a termination sequence. In some embodiments, a vector to be introduced into a Trichoderma host cell comprises a termination sequence and a promoter sequence derived from the same source. In some embodiments, the termination sequence and the promoter sequence are derived from different sources. One nonlimiting example of a suitable terminator sequence is the terminator sequence of cbh1 derived from a Trichoderma strain, such as a strain of Trichoderma reesei.

In some embodiments, the vector includes a selectable marker. Examples of suitable selectable markers include, but are not limited to, genes that confer resistance to an antimicrobial compound, e.g., hygromycin, phleomycin. Nutritional selectable markers may also be used, e.g., amdS, argB, pyr4. Markers useful in vector systems for transformation of Trichoderma are known in the art. (See, e.g., Finkelstein (1992) Biotechnology of Filamentous Fungi, Ch. 6, Finkelstein et al., eds., Butterworth-Heinemann, Boston, Mass.; Kinghorn et al. (1992) Applied Molecular Genetics of Filamentous Fungi, Blackie Academic and Professional, Chapman and Hall, London). In one embodiment, the selectable marker is the amdS gene, which encodes the enzyme acetamidase, allowing transformed cells to grow on acetamide as a nitrogen source. The use of A. nidulans amdS gene as a selectable marker is described in Kelley et al. (1985) EMBO J. 4:475-497 and Penttila et al. (1987) Gene 61:155-164.

An expression vector comprising a polynucleotide sequence encoding a catalase polypeptide may be any vector that is capable of replicating autonomously in a Trichoderma host cell or integrating into the DNA of the host cell. In some embodiments, the expression vector is a plasmid.

Methods of making a DNA construct comprising a polynucleotide encoding a catalase polypeptide and other sequences such as a promoter and a terminator, and to insert them into a suitable vector are well known in the art. Linking of polynucleotide sequences is generally accomplished by ligation at convenient restriction sites. If such sites do not exist, synthetic oligonucleotide linkers are used in accordance with conventional practices. (See, e.g., Sambrook (1989), supra; Bennett and Lasure (1991), supra, pp. 70-76.)

Introduction of Vectors into Host Cells

Introduction of a vector comprising a polynucleotide sequence that encodes a catalase polypeptide into a Trichoderma host cell may be performed using any of a number of well known techniques in the art, for example, transformation, electroporation, nuclear microinjection, transduction, transfection (e.g., lipofection or DEAE-Dextrin mediated transfection), incubation with calcium phosphate DNA precipitate, high velocity bombardment with DNA-coated microprojectiles, or protoplast fusion.

In some embodiments, stable transformants are produced, whereby the polynucleotide encoding a catalase polypeptide is stably integrated into the host cell chromosome. Transformants are then purified by known techniques.

In one embodiment, stable transformants including an amdS marker are distinguishable from unstable transformants by their faster growth rate and the formation of circular colonies with a smooth, rather than ragged, outline on solid culture medium that contains acetamide. Additionally, in some cases a further test of stability is conducted by growing the transformants on solid non-selective medium (i.e., medium that lacks acetamide), harvesting spores from this culture medium, and determining the percentage of these spores that subsequently germinate and grow on selective medium that contains acetamide. Alternatively, other methods known in the art may be used to select transformants.

In one embodiment, preparation of Trichoderma host cells for transformation involves the preparation of protoplasts from fungal mycelia. (See, e.g., Campbell et al. (1989) Curr. Genet. 16:53-56.) In some embodiments, the mycelia are obtained from germinated vegetative spores. The mycelia are treated with an enzyme that digests the cell wall, resulting in protoplasts. The protoplasts are protected by the presence of an osmotic stabilizer in the suspension medium. Such stabilizers include, for example, sorbitol, mannitol, potassium chloride, and magnesium sulfate. Typically, stabilizer concentration is in the range of about 0.8 M to about 1.2 M. In one embodiment, sorbitol is present in the suspension medium at a concentration of about 1.2 M.

Typically, uptake of DNA into a host Trichoderma cell is dependent upon calcium ion concentration in the uptake solution. Generally, about 10 mM to about 50 mM CaCl₂ is included in the uptake solution. In addition, the uptake solution also typically includes a buffering system (e.g., TE buffer (10 mM Tris, pH 7.4; 1 mM EDTA) or 10 mM morpholinepropanesulfonic acid (MOPS), pH 6.0) and polyethylene glycol (PEG). Although not wishing to be bound by theory, PEG may act to fuse the cell membranes, thus permitting the contents of the medium to be delivered into the cytoplasm of a Trichoderma cell and transfer of plasmid DNA to the nucleus. This fusion typically results in integration of multiple copies of plasmid DNA into the host chromosome.

Often, a suspension containing Trichoderma protoplasts or cells that have been subjected to a permeability treatment at a density of about 10⁵ to about 10⁷ per ml, typically about 2×10⁶ per ml, is used for transformation. A volume of 100 μl of these protoplasts or cells in an appropriate solution (e.g., a solution containing 1.2 M sorbitol 50 mM CaCl₂) is mixed with DNA. Generally, a high concentration of PEG is added to the uptake solution. For example, about 0.1 to about 1 volume of 25% PEG 4000 can be added to the protoplast suspension. In one embodiment, about 0.25 volume of 25% PEG 4000 is added. Additives including but not limited to dimethyl sulfoxide, heparin, spermidine, and potassium chloride may be added to the uptake solution and aid in transformation.

Typically, the uptake mixture is then incubated at 0° C. for about 10 to about 30 minutes. Additional PEG may then be added to the mixture to further enhance the uptake of the desired gene or polynucleotide sequence. For example, about 5 to about 15 volumes 25% PEG 4000 may be added. However, greater or lesser volumes may be suitable. The amount of added 25% PEG 4000 is often about 10 times the volume of the transformation mixture. After addition of PEG, the transformation mixture may then be incubated either at room temperature or on ice, followed by addition of a sorbitol and calcium chloride solution. The protoplast suspension is then added to aliquots of a growth medium, e.g., molten growth medium, which only permits growth of transformants.

### Production of Catalase

Expression of heterologous protein in Trichoderma is described, for example, in U.S. Pat. Nos. 6,022,725 and 6,268,328, Harkki et al. (1991) Enzyme Microb. Technol. 13:227-233, Harrki et al. (1989) Bio Technol. 7:596-603, EP 244,234, EP 215,594, and Nevalainen et al. (1992) “The Molecular Biology of Trichoderma and its Application to the Expression of Both Homologous and Heterologous Genes,” in Molecular Industrial Mycology, Leong and Berka, eds., Marcel Dekker Inc., NY, pp. 129-148.

Generally, Trichoderma host cells are cultured in a standard medium containing physiological salts and nutrients. Common commercially prepared media (e.g., yeast malt extract (YM) broth; Luria Bertani (LB) broth; Sabouraud Dextrose (SD) broth) may be used. Typically, cells are cultured at approximately 28° C. in shake cultures or fermenters until desired levels of catalase expression are achieved. Often, an inducing sugar such as lactose or glucose-sophorose is included in the medium.

When catalase is produced in A. niger, oxalic acid is produced as a side product at a level of about 30 g/l. It is necessary to remove most of the oxalic acid to avoid uncontrolled precipitation in the catalase product. Therefore, oxalic acid is precipitated from the A. niger fermentation medium with CaCl₂ as calcium oxalate. Production of catalase in T. reesei results in a much lower level of oxalic acid. The average oxalic acid concentration after four different T. reesei fermentations was only 1.24±0.15 g/l. Advantageously, this precludes the need for oxalate precipitation.

### Methods of Use

The invention provides a method for converting hydrogen peroxide to oxygen and water, comprising contacting hydrogen peroxide with a catalase enzyme produced in a Trichoderma host cell as described herein. Applications in which the methods of the invention may be used include, but are not limited to, removal of hydrogen peroxide after textile bleaching, pulp or paper bleaching, or use as a biocide. Nonlimiting examples of suitable process conditions for the catalase enzyme described herein include pH 3-9, 20-80° C., and up to 5000 ppm hydrogen peroxide.

### Compositions

The invention also provides compositions comprising a catalase enzyme produced in a Trichoderma host cell as described herein. Such compositions may be used in methods for conversion of hydrogen peroxide to oxygen and water.

In various embodiments, the catalase enzyme may be provided in a liquid, solid, whole broth liquid, or whole broth solid formulation.

In some embodiments, clarified catalase may be provided at pH approximately 3-9, with activity concentrations ranging from approximately 800 to 20,000 U/g. Liquid formulations can include polyols, such as 5-40% sorbitol or glycerol, salts, such as 5-20% sodium chloride, buffers, including citrate, phosphate, or acetate, and other ingredients, such as 0.01-2% formate and/or 0.01-2% sodium nitrite, and/or antimicrobial agents, such as potassium sorbate, sodium benzoate, parabens, and/or Proxel (1,2-benzisothiazolin-3-one).

### Kits

The invention also provides kits. In one embodiment, a kit provides catalase enzyme that has been produced in a Trichoderma host cell as described herein, optionally with instructions for use of the catalase enzyme, for example, in methods or applications such as, for example, removal of hydrogen peroxide after textile bleaching, pulp or paper bleaching, and/or use as a biocide. Suitable packaging is provided. As used herein, “packaging” refers to a solid matrix or material customarily used in a system and capable of holding within fixed limits components of a kit as described herein, e.g., catalase enzyme.

Instructions may be provided in printed form or in the form of an electronic medium such as a floppy disc, CD, or DVD, or in the form of a website address where such instructions may be obtained.

The following examples are intended to illustrate, but not limit, the invention.

### EXAMPLES

### Example 1

### Construction of pTrex3gM(CATE) Expression Vector

The plasmid pTrex3gM was produced by modification of and is smaller in size than pTrex3g (described in PCT Application No. WO 05/001036). pTrex3gM differs from pTrex3g primarily in the sequences responsible for plasmid replication in bacteria. pTrex3gM was constructed by amplifying the origin of replication and ampicillin resistance genes from pUC19 by polymerase chain reaction (PCR) using two primer pairs:

oMOB5
(SEQ ID NO: 5)
(GGTTCTAGAGGCCTAAATGGCCATGAGACAATAACCCTGATAAATGC)
plus
oMOB31
(SEQ ID NO: 6)
(AAGGCCTGCAGGGCCGATTTTGGTCATGAGATTATC);
and
oMOB51
(SEQ ID NO: 7)
(TCGGCCCTGCAGGCCTTAACGTGAGTTTTCGTTCC)
plus
oMOB3
(SEQ ID NO:8)
(GGTTCTAGAGGCCATTTAGGCCGTTGCTGGCGTTTTTCCATAGG).

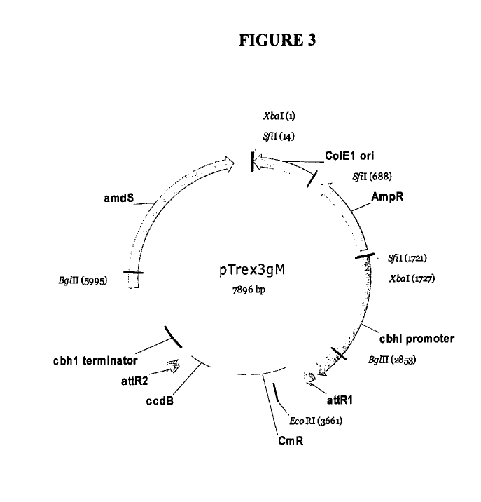

The two resulting PCR products were digested with PstI and XbaI and ligated with the 6.17 kb XbaI-XbaI fragment of pTrex3g to produce the Trex3gM expression vector. The structure of pTrex3gM is illustrated in FIG. 3.

The entire coding region of the catR gene (including introns) (SEQ ID NO:2) from the A. niger strain FS1 (GICC 20047) was amplified using high fidelity DNA polymerase PfuUltra II (Stratagene) with the primer pair:

oCAT 51
(SEQ ID NO: 9)
(CACCAAACAATAACAACAACAACAAACAACAACAACAACAACAACATG
CGTCATTTCTGGCTTTTGCCAG)
and
oCAT 3
(SEQ ID NO: 10)
(CGTGATACCCTTACTCATCCAGCGC).

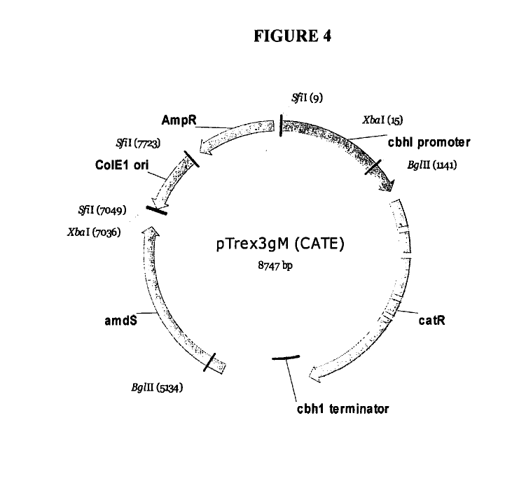

In addition to the complete coding region of the catR gene, the resulting PCR product contained an AC-rich 5′-untranslated region derived from the mycovirus PcV. A possible function of this region as a “translational enhancer” has been suggested (Jiang et al. (2004) J. Gen. Virol. 85:2111-2121. The PCR product was cloned into the pENTR vector (Stratagene) and subsequently transferred to the pTrex3gM vector using the Gateway® cloning system from Invitrogen, resulting in the vector pTrex3gM(CATE), illustrated in FIG. 4.

### Example 2

### Transformation of Trichoderma reesei and Screening of the Transformed Strains

Trichoderma reesei strain Morph 1.1 (pyr+) is a spontaneous pyr4 revertant of the quad-deleted RL-P37 strain (described in PCT Application No. WO 05/001036). Freshly-harvested spores from this strain were suspended in ice cold 1.2 M sorbitol, washed twice with the same solution, and subjected to electroporation with purified 7.04 kb catR-containing SfiI-SfiI fragment from the pTrex3gM(CATE) plasmid. The electroporation parameters were as follows: voltage: −16 kV/cm; capacitance: −25 μF; resistance: −50Ω.

Following electroporation, the spores were incubated overnight on a rotary shaker (30° C.; 200 rpm) in a medium containing 1 M sorbitol, 0.3% glucose, 0.3% Bacto peptone, and 0.15% yeast extract. The germinating spores were plated on a selective medium containing acetamide as a sole source of nitrogen (acetamide 0.6 g/l; cesium chloride 1.68 g/l; glucose 20 g/l; potassium dihydrogen phosphate 15 g/l; magnesium sulfate heptahydrate 0.6 g/l; calcium chloride dehydrate 0.6 g/l; iron (II) sulfate 5 mg/l; zinc sulfate 1.4 mg/l; cobalt (II) chloride 1 mg/l; manganese (II) sulfate 1.6 mg/l; agar 20 g/l; pH 4.25). Transformed colonies appeared in about 1 week. Individual transformants were transferred onto fresh acetamide selective plates and allowed to grow for 2-4 days.

Isolates exhibiting stable growth on selective medium were used to inoculate 0.17 ml of lactose defined medium (WO 2005/001036), p. 60) (NH₄)₂SO₄ 5 g/l; PIPPS buffer 33 g/l; Bacto Casamino Acids 9 g/l; KH₂PO₄ 4.5 g/l; CaCl₂*2H₂O 1.32 g/l; MgSO₄*7H₂O 1 g/l; Mazu DF204 5 ml/l; 400× Trace Elements 2.5 ml/l; pH 5.5; lactose (sterilized separately) 16 g/l. 400× Trace Elements solution: Citric acid (anhydrous) 175 g/l; FeSO₄*7H₂O 200 g/l; ZnSO₄*7H₂O 16 g/l; CuSO₄*5H₂O 3.2 g/l; MnSO₄*4H₂O 1.4 g/l; H₃BO₃ 0.8 g/l.) in wells of micro titer plates equipped with micro filters at the bottoms of the wells (Millipore MultiScreen-GV™). The plates were incubated for 4-5 days at 25-28° C. in an atmosphere of pure oxygen. The culture medium was separated by filtration and analyzed by polyacrylamide gel electrophoresis in the presence of sodium dodecylsulfate (SDS-PAGE). A new protein band was observed with slightly higher than calculated molecular weight of mature catalase polypeptide. This discrepancy may be due to glycosylation. The amount of protein in the catalase band exhibited strong clonal variation. The clone with the highest catalase expression was selected, allowed to sporulate on potato-dextrose agar, and purified through isolation of single spore subclones.

Catalase produced in the transformed T. reesei was catalytically active. For example, mixing a small (10 μl) aliquot of the culture broth from clone 329 (grown as described above) with 200 μl of 1% H₂O₂ caused immediate visible H₂O₂ decomposition accompanied by vigorous evolution of molecular oxygen (visible as bubbles). No such reaction was observed with culture broth from the parent strain Morph 1.1 (pyr+).

### Example 3

### Comparison of Expression and Distribution of Expressed Catalase in T. reesei and A. niger

### Sample Preparation

Catalase (A. niger catR) was produced in T. reesei as described in Example 2 and in A. niger as described in U.S. Pat. Nos. 5,360,732 and 5,360,901. 30 ml samples were centrifuged at 4000 rpm (3210×g) for 15 min. The supernatant was decanted and the pellet was washed with 50 ml deionized water. The washed cells were then centrifuged at 4000 rpm for 15 min, the supernatant decanted, and the pellet washed with 50 ml deionized water. The washed cells were centrifuged once more at 4000 rpm for 15 min, and the supernatant decanted. The cell pellets were frozen on dry ice. The average cell mass (dry cell weight) for A. niger was 43.3 g/kg, and the average cell mass for T. reesei was 43.9 g/kg.

### Chemical Extraction

Cell pellets were transferred to sample bottles and suspended in 40 ml deionized water. They were then incubated in a water bath at 30° C., with moderate mixing with a magnetic stirrer. 25 ml of extraction stock (50 g/l NaCl; 6 g/l potassium sorbate; 12 g/l sodium formate; 12 g/l acetic acid; about 6 g/l sodium hydroxide added to adjust pH to 4.8-5.0; 0.1 g/l protease-C) was added to added, then the pH was adjusted to 4.8-5.2. The sample bottle tops were loosely closed, then incubation proceeded for 72-84 hours. The resulting sample solution was transferred into a 100 ml beaker and the volume measured. Catalase activity was then determined using the Baker Catalase Assay procedure.

### Baker Catalase Assay

The Baker catalase assay is a “double exhaustion” analysis based on the simultaneous inactivation of catalase by hydrogen peroxide and the breakdown of hydrogen peroxide by catalase. In practice, residual hydrogen peroxide is analyzed in the reaction mixture after incubation with catalase for 60 minutes at pH 7.0 and 25° C. One Baker unit is defined as the amount of catalase that will decompose 264 mg of hydrogen peroxide under the conditions of the assay.

### Preparation of Reagents

0.2 M Phosphate buffer, ph 7.0: 10.76 g NaH₂PO₄ and 17.32 g Na₂HPO₄ were diluted into 800 ml distilled water and mixed well. The pH was checked, and then the solution was adjusted to a final volume of 1000 ml with distilled water.

Buffered substrate: 500 ml of 0.2 M phosphate buffer, pH 7.0, was mixed with 450 ml deionized water. 44-46 ml of 30% hydrogen peroxide was added. This solution was stable for one week at 4° C.

M Sulfuric acid: 55.6 ml concentrated H₂SO₄ was diluted into 900 ml of distilled water, mixed well, and allowed to cool. The final volume was adjusted to a final volume of 1000 ml with distilled water.

40% (w/v) Potassium iodide solution: 200 g potassium iodide was added to 250 ml distilled water and mixed well. The volume was adjusted to 500 ml with distilled water. The solution was stored in an amber bottle under refrigeration (4° C.), and discarded after one week.

1% (w/v) Ammonium molybdenate solution: 1.0 g (NH₄)₆Mo₇O₂₄.4H₂O was added to 90 ml distilled water and mixed well. The volume was adjusted to 100 ml with distilled water.

0.25 M Sodium thiosulfate solution: 62 g Na₂S₂O₃.5H₂O was added to 900 ml distilled water and mixed well. The volume was adjusted to 1000 ml with distilled water. This solution was stored no longer than one week.

### Assay Procedure

Samples were assayed in triplicate as follows:

1. Pipette 5 ml of 1.0 M sulfuric acid into a 100 ml Erlenmeyer flask.
2. Pipette 4.0 ml of buffered substrate into the sulfuric acid and add 5 ml of 40% potassium iodide solution. Swirl to mix.
3. Add two drops of 1% ammonium molybdenate solution and titrate immediately with 0.25 M sodium thiosulfate solution.
4. The titration end point is reached when the free iodine color disappears completely. Color may return after a few minutes and should be ignored.
5. A titration volume of 14 to 16 ml should be obtained. Adjust the concentration of the hydrogen peroxide in the buffered substrate solution, if necessary, to obtain this titration volume.
6. Dispense 50 ml aliquots of the buffered substrate into lightly stoppered 100 ml Erlenmeyer flasks and pre-incubate in a water bath set at 25° C. for 15 minutes.
7. Dilute catalase samples to between 5-9 Baker units/ml with 0.2 M phosphate buffer.
8. Pipette 200 μl of diluted catalase samples into the pre-incubated 100 ml Erlenmeyer flasks at 3 minute timed intervals and mix immediately by swirling.
9. Incubate at 25° C. for 60 minutes. Shake occasionally to liberate oxygen from the reaction mixture.
10. At the end of the incubation period, shake the reaction mixture until all of the oxygen has been removed.
11. Analyze residual hydrogen peroxide by using the method described above in steps 1-4. (Pipette 4.0 ml of test solution into the sulfuric acid and follow steps 2-4.)
12. A substrate blank, using 200 μl of 0.2 M phosphate buffer instead of diluted catalase sample in step 8, should be similarly incubated and analyzed. (The blank should require 14-16 ml titration volume.)

### Calculation of Activity

Catalase activity in Baker Units (U/ml)=(B−S)×DF
B is the titration volume of the blank (ml)
S is the titration volume of the sample (ml)
DF is the dilution factor of the sample
   For greatest accuracy, the difference (B−S) should be between 5-10 ml.

### Results

The results are shown in Table 1.

TABLE 1

| | Mycelia Activity | Total Activity | Intracellular |
|---|---|---|---|
| | (U/ml) | (U/ml) | Activity (%) |
| A. niger catalase | 568 | 926 | 61 |
| T. reesei catalase | 401 | 1429 | 28 |

### Example 4

### Comparison of Tm for Catalase Expressed in A. niger and T. reesei

Catalase (A. niger catR) was produced in T. reesei as described in Example 2 and in A. niger as described in U.S. Pat. Nos. 5,360,732 and 5,360,901.

### Effect of pH on Melting Point

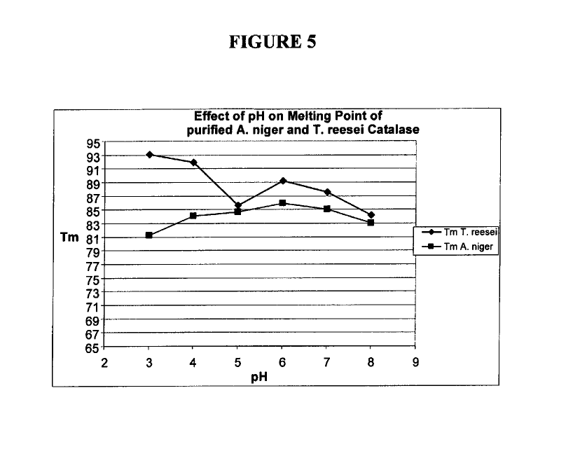

Catalase expressed in A. niger or T. reesei was suspended at a concentration of about 0.5 mg/ml in buffers adjusted to pH 3-8, in 1 unit increments. Buffers were 250 mM citric acid pH 3, 250 mM sodium acetate trihydrate pH 4, 250 mM sodium citrate pH 5, 25 mM bis tris propane, pH 6, 7, and 8. Samples were analyzed by differential scanning calorimetry (DSC), using a MicroCal VP-Capillary DSC (MicroCal, LLC Northampton, Mass.), run with a temperature scan from 30° C. to 120° C. at a rate of 200° C./hour. The same buffer without protein added, was used as the reference solution. Melting point (Tm) was determined by observing the maximum peak height from a plot of Cp (cal/deg C) vs. temperature. The error of DSC for this measurement was approximately +/−1° C. The results are shown in FIG. 5.

### Effect of H₂O₂ Pretreatment

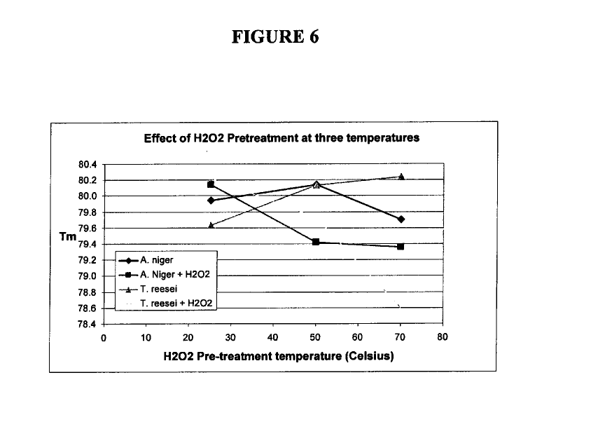

Catalase expressed in A. niger or T. reesei was suspended at a concentration of about 0.5 mg/ml in buffer (50 mM potassium phosphate, pH 7) that had been pre-warmed to 25° C., 50° C., or 70° C. H₂O₂ was then added at a concentration of approximately 3%. Samples were held for 15 minutes at their respective temperatures, then cooled to 4° C., and analyzed by DSC using the same method as described above. A parallel set of samples that were treated the same, except not exposed to H₂O₂, served as the experiment controls. The error of DSC for this measurement was approximately +/−1° C. The results are shown in FIG. 6.

### Example 5

### Expression of A. niger Catalase in T. reesei Host Cells with Endo T Deletion

Construction of a Disruption Cassette for the Endo T Gene of T. reesei.

Endo T gene was identified in the genomic sequence of T. reesei (http://genome.jgi-psf.org/Trire2/Trire2.home.html) using information provided in PCT Application No. WO 2006/050584. Its 5′ flanking region (1.9 Kb) was amplified by PCR using primers SK915 (5′-CTGATATCCTGGCATGGTGAATCTCCGTG-3′) (SEQ ID NO:11) and SK916 (5′-CATGGCGCGCCGAGGCAGATAGGCGGACGAAG-3′) (SEQ ID NO:12). The 3′ flanking region (1.7 Kb) was amplified by PCR using primers SK917 (5′-CATGGCGCGCCGTGTAAGTGCGTGGCTGCAG-3′) (SEQ ID NO:13) and SK918 (5′-CTGATATCGATCGAGTCGAACTGTCGCTTC-3′) (SEQ ID NO:14). PfuII Ultra (Stratagene) was used as the polymerase in all PCR reactions.

The products of the PCR reaction were purified with the QIAquick PCR purification kit (Qiagen) by following the protocol listed in the manual. Both amplified DNA fragments were digested with restriction endonuclease AscI, followed by purification of digested DNA using QIAquick kit. The two DNA fragments were mixed and used as a template for a fusion PCR reaction with primers SK915 and SK918. The product of this reaction, a 3.6 kb DNA fragment was cloned into pCR-Blunt II TOPO vector using the Zero Blunt TOPO PCR Cloning Kit (Invitrogen). The structure of the resulting plasmid (pCR-BluntII-TOPO (5′-3′ flank)) was confirmed by restriction analysis.

A mutant form of the T. reesei acetolactate synthase (ALS) gene conferring resistance to chlorimuron ethyl (WO 2008/039370) was amplified using PCR primers SK949 (5′-GTTTCGCATGGCGCGCCTGAGACAATGG-3′) (SEQ ID NO:15) and SK946 (5′-CACAGGCGCGCCGATCGCCATCCCGTCGCGTC-3′) (SEQ ID NO:16) and pTrex-Glucoamylase vector (WO 2008/039370, Example 2) as the template. The product of the PCR reaction was purified with QIAquick kit, digested with AscI, purified again and ligated with pCR-BluntII-TOPO (5′-3′ flank) digested with the same enzyme and purified similarly. The orientation of the insert in the resulting plasmid pCR-BluntII-TOPO (5′ flank-ALS marker-3′ flank) was established by restriction analysis.

An additional fragment of T. reesei chromosomal sequence (referred to as “3′-repeat”) was amplified using the same techniques and primers MC40 (5′-CTATGACATGCCCTGAGGCGATGCTGGCCAGGTACGAGCTG-3′) (SEQ ID NO:17) and MC41 (5′-CAGCCTCGCGGTCACAGTGAGAGGAACGGGGTGAAGTCGTATAAG-3′) (SEQ ID NO:18). This sequence is located on T. reesei chromosome further downstream of the 3′-flank area that is contained within pCR-BluntII-TOPO (5′-3′ flank). The 0.46 kb product of this PCR (3′-repeat) was cloned upstream of the ALS gene in the pCR-BluntII-TOPO (5′ flank-ALS marker-3′ flank) using In-Fusion Dry-Down PCR Cloning Kit (Clontech). pCR-BluntII-TOPO (5′ flank-ALS marker-3′ flank) was digested with Pas I and BstEII for use as a vector to clone in the 3′ repeat. The resulting construct pCR-BluntII-TOPO (5′ flank-ALS marker-3′ repeat-3′ flank) was used as the template for a PCR with primers SK1008 (CTAGCGATCGCGTGTGCACATAGGTGAGTTCTCC) (SEQ ID NO:19) and SK1009: (CTAGCGATCGCGCAGACTGGCATGCCTCAATCAC) (SEQ ID NO:20).

The 7.5 kb DNA product was cloned into pCR-BluntII-TOPO vector using the corresponding kit from Invitrogen. The resulting plasmid was digested with AsiSI and a 7.5 kb DNA fragment (the Endo-T deletion cassette) was purified by preparative agarose gel electrophoresis. The complete nucleotide sequence is shown below.

(SEQ ID NO: 21)
5′-CGCGTGTGCACATAGGTGAGTTCTCCGAACAAACTGTGCGCAAAGA
GTCACAGGGAATTGACGAGATGACAGGGTGGCAGCACGCCCACCGAGAC
CGTCTCATCGCAACTACGACTTGCCCGCCACTATGCATTACAAGCTCTT
CTCGGGGGTATCAAGGATCCGGCTGAAGCACTGGAGGTGAGTTAACGAC
GCCCTTGACAGGACGTGACATTTGCTTATACAATGGCACCTCGACTGGA
GCAGGCTCAACGGAGGCTGTACAAAACGGCGTCGGTGTACATGAATCTT
TGTTGGCAGTTGGCTGATATTAACGTCCTCTATTCCGTTCATCGCTCCC
ACGAACGCGAAGAACGGGCTATGATGCGCAAGGAGATGGAGCAGGCAGA
AAGGAAAGAGAAGCGGAAGGGCAAGAAGCCGAAGCATCGTGGCAACAAG
ACAGACAGCGGCAAAGGAGCTGCCGGGAAAAGTGACGGCGAAACCGCAA
CGGAGCTACCGGTTAGTGACACATATCCCAGCGGATTCGGCATCCAGCT
CGCCCACGTCGTCCTCTTACGCAAAATCTGGAGGCAGAATCAGACGACC
CTGGTGCAGCTCGAGGCCCAAAGCGTGCTCCTGTCTGCCCTCATGAGGG
GGGAGGTGCAAGCCCCCTCGCTTCTGTCTTCAGCGTATGAAGAAATGGT
CAAGTCGGTAAAGCTGTCGTGCGAGGTCTTGGAGAAGCTCTTGGAGAAA
TTCGAGGTGCAACCTCATGGTTTGAATATCCCGGACAAGCTGAGGAAGG
CTGCGATGGAGCTATAGGCACGGGCGGCAGTACTCTCTCTCTCTCTCTG
CTCTCACTTTTGCTCTACTCACTCTCGGCCTCTGTCTATTTCCGTCTGC
CTCGCATAGTCTTTCCCTGATTCTTTTTCTTTCACCGATGGGACATCTT
GTATCTAGTGTGAGACATTGATATTGTAGCGAAACAGCGAGGTAGTATT
TCCAAGACAGGATAGAGTCCAGACCCCGGTGCTCCAACACTTGCAGTCC
AAGACTTACAAGGATCACGGCCAACCGAGGACCGGGATTGGTGGGCAGG
TCTCGAGCGTCCAGTGCGATCGATCTATATCATTGTCTCACGCCGCTCT
CGGGCAAGCAAACCGTCGCTTTGTGATGCTTGGCCTTAATACAGTAGTA
GTAGTAACGCAGGTGCGAAGAATGCCGGCCATGTTGCCCCCCTGTTGCC
TCTGTTTATGTACGAGAACTACTAGCCAGCCATGGCGATGGCGCCCCGG
CGGACGGGCGCCATTGTCGAGGTTAAAGTTGGCGGCGACGGCTAATTGG
ACTCTGGACCACAAGGTTCAAGCACGAGATGCAACCTTGGCCCAAGCAT
AAACCGGCCTCGTCCTATCAGACGACAGCCGGCTCTTCCTTGCCCATCT
ACTCCCTCATTACTCGGACGGATGAGACCCGGCAAAAGCGCGTGTTTCT
TATTTTGTTTTCCATGTCTATTTCTTCTTCTTCTTCTTCTTCTTCTTCG
TCCGCCTATCTGCCTGGCGCGCCTGAGACAATGGCCGGCAATGGTAAAA
AGGACCAAGATGTACTAGGTAGTTGCAATGTGGCTTATTACCTACCTAC
TACCTGGTAGGCACCTACTAGGTACTTGGGTAGACGGACAATGAAATTT
GAAGTCGGGGTTGCAGGAAAGCAGGGCGCTGGACACATTGTGCTTCAGG
CGGTACCCGTCGTCATCGTCAGCCAATGTCGAGGCCCGGCAGCCCGAGG
AGCGAGACAACCTTGGCCGGAGGAGCCCGCAGGTACCTGCCAAAGCGCG
GCTGGTACCTCTCAACCCTCTCAGGCCTGTTGGATGCCCTATGACATGC
CCTGAGGCGATGCTGGCCAGGTACGAGCTGGACGTGTCGTTGAGGACTC
CCTGGGCCCTGAAGTATTAAGATTGTCAGTGGATGAACTTGTCGTCATC
TTCATCATCATCAGTGATGAATAAGTGTCAGTCTGCTCACCATGAGGCC
GTGGCTGCGTTGAGATAGTGCTGGGCCACGTAGGCGACGGTTGCGTGGC
CCAGGACTCCCCAGGCGTGGGCGCCCTGGAGCGTGGCCAGAGTCGCCAA
GGCAATCTTTGACATGGAAGGCATTATGTTGAGGCGAGTCTTGAGAACC
GGGATCCAACTACTGGCTCGACTTTGACTGTTGCGTAGATGGAGTCAAG
ATTCCTCACTGATATCACGGAGACTCAACGACGACGAGGAACAAGGACG
GGCATCGCCATCTTATACGACTTCACCCCGTTCCTCTCACTGTGACCGC
GAGGCTGCTGATTGGCTGGTTGCCACGGGCTGGGCGGTCCCTGAAGTTG
TTGCCATCTGAACTCTGTCGGCGCTGGCGTCGGCTGCGCCCAATGGGAG
GCGAGACAACTCAGGGTACTAGAATCACTGACAGAAGAAGAGAATCGAA
AGTAGGTAGACAGCCAATTCGTTGCATGGCAGGCAACCGCACAGGAGAA
AAATTGACTACCCCACAATCAGGCACAGTAAGTAGGGCACAGTACGTAT
GTACAGACAAGGCGCAAGCGATACTGCGCGACCCGGTACCTCGCCGGCT
TGACACGTGCGACAGGCTACTTTACTAGTATTCGCAGCGGCGGGTCGCG
CATTATTACATGTACTGTGCCGCCATTTGATGACTGGGCTGCTGCAGTA
TTAGTAGATCTGCCCGGCATCGCCCTTCCATGGGCGCGACCCGGGACTG
GACCCTCTGACTCTACCTACATGTACCTAGGCCGGGCCGGGCTTGGTGA
CTTTTGTCCGATCAGGTCGTTCGCCTGGCTACCTATTATTTCTCTTTCT
TCTTCTCCATCCTGCTTCTGGCCTTGCAATTCTTCTTCGCCACTCCTCC
CTCTTCCCCCCGCGATACCCTTGAATTCGTCAGAGAGGAAAAGACGAGA
AAAAAAAGGGCAGCAGAGACGTCGGTCTGGCTCACGTGCTGCATCTCTG
CGCACTCTCATTTTTTTTATTGTCCGACCCCTCCCTCAACCTTCTCCTT
CGTTGACAGGCTAAGCCTTGCTTCGACGCTCTCTCTTTGAATTTTTCTA
CTTCTACCTTCTTTTCTTGCGTGTTACCCACCATAGCTCGATTCACGAT
GCTCCGAAGTCGCCAAGTCACAGCCAGGGCCGTCCGGGCTCTGGGCCAG
GCGCGCGCCTTTACCTCGACGACCAAGCCTGTCATGATCCAGAGCAGCC
AGAGGAAACAGGCCAACGCCAGCGCTGCTCCGTAAGTCGCCCATTGCCA
TTGCATCTTCTGTTTGATATATACTTCCTGCTGCTTGCGTGGCGTCGTC
TCTCGGTTATGCGTGTCAAGGACCAGGTGTGTTCGCATCGTGGTTTTCC
AGCGCCGATTACCGGGGGACGAATTTTTGGCTGCTCAACTCGCGCGCGC
GCATTCTGATTCTTCGTTTTCAATCTTGAGCGACAACTGGCTAACATAA
TGGCCATTGGCAATTGCTTCACACAGACAAGTCCGCCCTGTACCGAGCC
CTGCTTTCAACGCTGAAGACAAAGACCGCAGCCATGTGCAGCCTCTGGT
CAACCCGTCGAAGCCCGACATGGATGAATCGTATGTCCACGTCCCCTCG
TCCCGCCCTACAAAATGAACACGATTACACCAGAATTTTTGCAACAATC
GACACTTCTATAACAGACCAATTGAGCTTTGTTCTGACCAATCATGTTG
CTCTAGATTCATTGGCAAAACCGGAGGCGAAATCTTCCACGAGATGATG
CTGCGACAGGGTGTCAAGCACATTTGTAGGTTCCGATGCCGGCCGCCCA
CACGGGCTCCATCCTTGCTCCATCTCTCCAGCTAGGCAAATCTCGCTAA
CCTTGAGTCACCATCCAGTCGGATACCCTGGCGGCGCTATCCTGCCCGT
CTTCGACGCCATCTACAACTCAAAACACTTCGACTTCATCCTGCCCCGT
CATGAGCAGGGAGCTGGCCATATGGCCGAGGGCTATGCCCGTGCCTCGG
GCAAACCCGGTGTCGTCCTGGTGACTTCCGGCCCCGGTGCTACCAATGT
CATCACGCCCATGCAGGATGCCCTGTCGGACGGAACGCCCTTGGTCGTC
TTCTGCGGCCAGGTCCCCACCACGGCCATCGGCAGCGATGACTTCCAAG
AGGCCGACGTCGTGGGCATCTCGCGGGCCTGCACCAAGTGGAACGTCAT
GGTCAAGAGCGTTGCTGAGCTGCCGCGGAGAATCAACGAGGCCTTTGAG
ATTGCCACCAGCGGCCGCCCTGGCCCCGTCCTCGTCGACCTGCCCAAGG
ATGTCACGGCTGGTATCCTGAGGAGAGCCATCCCTACGGAGACTGCTCT
GCCGTCTCTGCCCAGTGCCGCCTCCCGCGCCGCCATGGAGCTGAGCTCC
AAGCAGCTCAACGCCTCCATCAAGCGTGCCGCCGACCTCATCAACATCG
CCAAGAAGCCCGTCATCTACGCCGGTCAGGGTGTCATCCAGTCCGAGGG
CGGCGTTGAGCTCCTGAAGCAGCTGGCGGACAAGGCCTCCATCCCCGTC
ACCACCACCCTCCATGGCCTGGGTGCCTTTGATGAGCTGGACGAGAAGT
CGCTGCACATGCTGGGCATGCACGGCTCGGCGTATGCCAACATGGCCAT
GCAGCAGGCCGACCTCATCATCGCCCTCGGCAGCCGATTCGACGACCGT
GTTACTCTGAATGTCTCCAAATTTGCGCCTGCAGCCAGGCAAGCTGCTG
CCGAGGGCCGCGGCGGCATCATTCACTTTGAGATCATGCCCAAGAACAT
CAACAAGGTCATCCAGGCGACCGAGGCCGTCGAGGGCGACGTCGCCACC
AACCTGAAGCACCTCATTCCCCAGATTGCCGAAAAGTCCATGGCGGACC
GAGGAGAGTGGTTCGGCCTCATCAATGAGTGGAAGAAGAAGTGGCCCCT
GTCAAACTACCAGCGCGCGGAGCGGGCTGGCCTCATCAAGCCGCAGACG
GTCATGGAGGAGATTAGCAACCTGACGGCCAACCGAAAGGACAAGACGT
ACATTGCCACGGGTGTCGGCCAGCACCAGATGTGGGTTGCCCAGCACTT
CCGCTGGAGGCACCCTCGATCCATGATTACCTCTGGTGGTCTGGGCACC
ATGGGCTACGGTCTCCCCGCGGCCATTGGCGCCAAGGTGGCCCAGCCCG
ACGCTCTCGTAATTGACGTTGATGGCGATGCCTCGTTTAACATGACGCT
GACGGAGCTGTCGACTGCTGCACAGTTCAACATTGGCGTCAAGGTGGTT
GTGCTCAACAACGAGGAGCAGGGCATGGTGACGCAGTGGCAGAACCTCT
TTTACGAGGACCGATATGCCCACACGCACCAGAAGAACCCCGACTTCAT
GAAGCTGGCCGACGCCATGGGCGTTCAGCACCAGCGCGTGACGGAGCCG
GAGAAGCTGGTCGATGCCCTGACGTGGCTGATCAACACCGATGGCCCGG
CCCTGTTGGAGGTTGTCACGGACAAGAAGGTGCCTGTCCTGCCCATGGT
GCCCGCCGGATCGGCCCTGCACGAGTTCCTCGTCTTTGAACCTGGTGAG
TCTACTTCAGACATATTGCTTGCGCATTGCAGATACTAACACTCTCACA
GAAAAGGATAAGCAGCGCCGTGAGCTGATGAAGGAGAGAACAAAGGGTG
TGCACTCCTAAAGCGATGATGTCTGCGAGGGGTTCTTCGTTGAACCCTA
GTTCAGGCACCATCTTACCCTCTTATTTTTTCCCGTGGGCTTTCATTTT
GTGTCATCCGAGCATGACGTTGTAGGGTTGGAGTTTCTTCCTTTTTATC
TTGTCATTTACTGGTACCCATAGGCGCGAGACTAGGCTTCCATGTTTTG
TTTTGCGACTTTCAAAAAGTACTTTTAGTGGTTTGGGGCACGACGAGGG
GGGGCAACCTCTTCTGTCGAAAAAGGTGGCTGGATGGATGAGATGAGAT
GAGATGAGGGTGAAGATAGATACCTGCAGTGTTTTTGACGCGACGGGAT
GGCGATCGGCGCGCCGTGTAAGTGCGTGGCTGCAGGGGGTTGCAGCATG
GCGCGCAAGGTTGCATCAGCTGAGCGACGATCGAGGAAAGGATCTGGAC
GATCATGGACGGATGTGGGATCAAGGTCGTGGAGATGTGTATCTATGTA
TACATTTTGAGTAGTGAAACGGGCGAGGCTCGTGTTGGTGTTGGGACTT
GTCATCTTCCTATAGTACAAGGTTATACATAGGTAGGTAGGTAGGTAGG
GAGGGACTGGGTGAGTGGCATGTGATGGCTGTATGCAAGTCCTAGTAGT
ACACGGCACATGACAGAGCCTCCCTGTGTGTGTGCTCGACCATGCATCA
TCAGCCTCATAGTGCAGATCGTGCTCGTGTCTTCTCCCTCTCGCTCTTC
ATGAACGCCCACCATGCCGATTCCCGCCATCCGTTCAAAGTCGCGGGAT
ACACGGGCATCTTCCCATCCAGCGTGGCATCATGCGCCAGGGCCACAAA
CACGGTGTCGTCCTGGTCCAGCTTCCGAAGCCTCTCCATGGACTTGATC
GCCTCGTCCACGTCCTGGTGCATGCTGGTCCCGCCTGGACACCCGTCGA
GGCTGAGCGGGCGGCTTCCATCGAGAACACTGCGAGCGTGGCAGCAGTC
GCCGCCCATGAGGACCCATTCGTCGAAGCCCGTCTGGGCCAATCCGGTG
ACGTGGCCGGGGAGGTGTCCCGGCGCGTCGACAATGTAGAAGCTGCCGT
CGCCGAAGAAGTCGTGAGCATACGGGAACGGGCCGAGAGGAATCCATTT
GTCCTCGGTCCGGCTCAGCTCGGTGTAATTCTCGCGGGTGAGCAGCGAT
CTCAGCATCGCCGAGTTGGGGTCGACGGGGTATCCGGGCAGGGCGGCTG
CTTTTGTGCCCGGGCCGGCGATGTAGGGGACGTTGGGGAAGAGAGAGGG
ATCGCCGATGTGGTCGTAGTGGATATGGCTGACGACGATTGCGCTCAGG
GAGTCTGGGGAGACGGGGCCCTCGGAGAGGAGGTCGCGGGCGTCTTTGG
GGACTTGGGGTTCGAAGATGTGGCTGATGGAGCGGAAGAGCGGAGGGAT
GACGTCCAGGTTCTGGAGAAATGGTTATTTGAGATTTAATGTCCGGAGA
TGGAATTTCTCGTCGGTAAATGGACTCTGCGTGAGCCTTGAGGACACTC
ACCTTTGGCAGGCCGGCGTCAAACAAGATGTTCTTCCCCGAGGGATGGC
GCACGAGGAAGCTGTAGTCGGGAAGACGCTGGCGCACCTTGACAATGTC
GTCGTCGCCGTCTTGCCAGATCCATCGGTCGGGCAGGTGAACCCACCCC
GTGGGAAGGGCGTATACCTGGACAGTGTTGGTCGAGGCAAACGAGGATA
GGAGACCCATGATGAAGACGGGATCCCAACTGCAACGGAACGGATGGGG
TGATTGAGGCATGCCAGTCTGCGCGAT-3′

Disruption of the Endo-T Gene in T. reesei.

A quad deleted strain of T. reesei (Δcbh1, Δcbh2, Δegl1, Δegl2) is described in PCT Application No. WO05/001036 This strain was transformed with the deletion cassette listed as SEQ ID NO: 21 using the transformation method described by Penttilä et al. (Penttilä M. et al. 1987. A versatile transformation system for the cellulolytic filamentous fungus Trichoderma reesei. Gene 61: 155-164). The transformants were selected on a Modified Vogel's medium containing 200 ppm chlorimuron ethyl (WO 2008/039370). Transformants were cultured in liquid medium and culture supernatants were analyzed by SDS gel electrophoresis.

Two clones (#11 and #74) displaying an upward shift in mobility of most of the protein bands on the gel were identified. Chromosomal DNA was isolated from these two strains as well as the parent quad deleted strains of T. reesei. PCR analyses were performed on these DNA preparations using primer pairs MC 42 plus MC 48 (5′-CTCGCCATCTGACAACCTACAAATC-3′ (SEQ ID NO:22) and 5′-CTAGTACCCTGAGTTGTCTCGCCTCC-3′) (SEQ ID NO:23) and MC 45 plus MC 50 (5′-CCTCTACCATAACAGGATCCATCTG-3′ (SEQ ID NO:24) and 5′-CGTGAGCTGATGAAGGAGAGAACAAAGG-3′) (SEQ ID NO:25).

Products of the expected size (2.9 and 2.3 kb) were obtained with DNA isolated from clone #74. This clone was subjected to two successive rounds of purification (by isolation of progeny of a single spore). DNA was isolated from the purified transformant #74. PCR analyses were repeated confirming successful deletion of the Endo-T gene.

Transformation of the Endo-T Deleted Strain of T. reesei with Catalase Expression Vector

Freshly harvested spores of endo-T deleted strain of T. reesei were suspended in ice-cold 1.2 M sorbitol, washed twice with the same solution and subjected to electroporation using purified 7.04 kb SfiI-SfiI fragment of the plasmid pTrex3gM(CATE). Transformation and screening of transformants were done essentially as described above. Clone #26 expressing high amount of catalase was identified and subjected to two rounds of spore purification Catalase produced by a purified isolate of this clone was used in all studies on the effect of Endo-T deletion on the properties of the recombinant enzyme.

### Example 6

### Comparison of Catalase Expressed in A. niger, T. reesei, and T. reesei with Endo-T Deletion

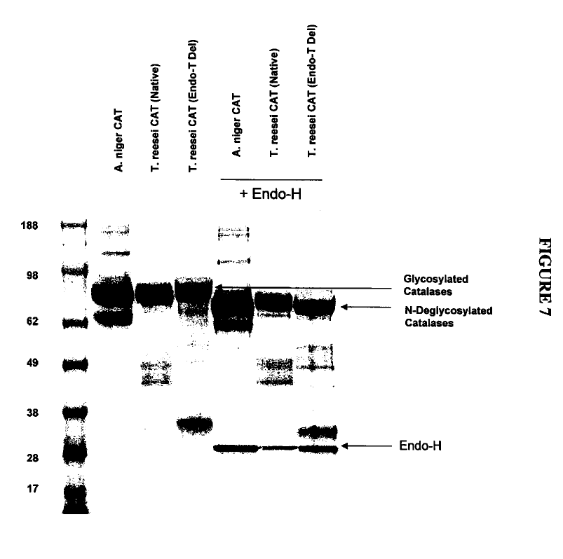

A. niger catalase was expressed in A. niger, T. reesei, and T. reesei with Endo-T gene deletion. Samples of each catalase were digested with Endo H enzyme to effect deglyosylation. Samples with and without deglyosylation were run on an SDS-PAGE gel. The results are shown in FIG. 7.

The change in molecular weight before and after Endo H treatment to remove glycosylation seems larger for the samples from A. niger and T. reesei with Endo-T deletion. Thus, it appears that these catalase enzymes contained higher levels of glycosylation than catalase from T. reesei without the Endo-T deletion.

The starting molecular weights of the catalase enzymes from A. niger and T. reesei with Endo-T deletion seems slightly higher than the starting molecular weight of catalase from T. reesei without the Endo-T deletion. Thus, these catalases appear to contain a higher degree of glycosylation than the catalase from T. reesei without the Endo-T deletion. This difference could alternatively or additionally be due to sequence differences, but this has not been investigated.

### Example 7

### Activity Comparison of A. niger Catalase Produced in A. niger, T. reesei, and T. reesei with Endo-T Deletion

### Evaluation of Catalase

Evaluation of catalases was based on the observation that different catalases demonstrated different performance. These differences were most evident under ‘stress conditions’ (high temperature and a high concentration of hydrogen peroxide). Each catalase was dosed in the same total amount of activity units for this evaluation. The total activity dosed was chosen such that the hydrogen peroxide was degraded in around 20 minutes.

### Determination of Catalase Activity

Enzyme activity was determined using the following method.

### Reagents

Substrate Solution:

Mix—25 ml 0.2 M Phosphate buffer pH 7.0 with
130 μl of 30% H₂O₂ and
make up the volume to 100 ml with demineralised water.

The absorbance of the substrate solution should be between 0.52 and 0.55. Adjust the amount of peroxide if needed.

Buffer solution: Dilute 25 ml of 0.2 M Phosphate buffer pH 7.0 to 100 ml with demineralised water.

### Catalase Sample Preparation:

Accurately weigh (4 decimals) around 1 gram of the catalase product in a 10.00 ml volumetric flask, make up the volume to 10.00 ml mark and mix. From this solution further dilutions can be made.

### Determination of Enzyme Activity:

Add to a quartz cuvette:
2.9 ml substrate solution
100 μl (diluted) catalase sample and mix

Measure the time for the absorbance at 240 nm to go down from 0.450 to 0.400 (in seconds). The time should fall into the range between 35 and 50 seconds. If the time falls outside this range test a different dilution (trial and error) until the right dilution is found.

### Calculation of the Catalase Activity:

The activity of the catalase product can be calculated with the following formula.

$$Activity U \text{/} g = \frac{1.15 µ mol \text{/} ml \times 3 ml \times 60 s \text{/} min \times F \times V}{T \times 0.1 ml \times w} = \frac{2070 \times F \times V}{t \times w}$$

$$F = dilution factor$$

$$V = volume of the volumetric flask$$

$$t = time ( s )$$

$$w = weight of the catalase product ( g )$$

### Catalase Performance Evaluation

### Reagents

Substrate solution (1000 ppm H₂O₂):

Mix 250 ml—0.2 M Phosphate buffer pH 7.0 with
3 ml 30% H₂O₂ and

Make up the volume to 1 l with demineralised water.

Buffer solution: Dilute 250 ml of 0.2 M Phosphate buffer pH 7.0 to 1 l with demineralised water.

### Sample Dilution:

The catalase dilution needs to be done in buffer. The goal is to degrade the hydrogen peroxide in around 20 minutes. For Aspergillus niger catalase, which was used as a reference, around 400 units were used. For other catalases, the same amount of units were dosed.

### Procedure

The incubations were performed in Schott bottles of 250 ml volumes. 100 ml of substrate was added to a bottle and this was placed into a water bath at 70° C. When the substrate temperature was 70° C., the reaction was started by adding a small volume (1 ml or less) of the (diluted) catalase to the bottle and at the same time, a timer was started. The solution was mixed from time to time. At 1- or 2 minute intervals, 3 ml was transferred from the bottle into a test tube containing 500 μl of a 10% sulphuric acid solution to stop the reaction. Sampling was continued until to take samples until 15-30 minutes. For t=0 measurement, 3 ml of substrate was transferred into a test tube containing 500 μl of a 10% sulphuric acid solution. For a blank test (to test the stability of the peroxide solution under the test conditions), buffer was added (same volume as in the test with catalase) to a bottle with substrate and the same procedure was followed. The absorbance of the solutions was measured at 240 nm.

Evaluation of the Performance of Aspergillus niger Catalase and Aspergillus niger Catalase Expressed in Trichoderma reesei

### Samples

A. niger catalase expressed in A. niger and T. reesei were tested in the activity assays described above.

### Sample Preparation

1 g of the catalase was weighed in a volumetric flask of 100.00 ml volume and brought up to 100.00 ml using the 50 mM phosphate buffer. 1.2 ml was used for A. niger and 1.39 ml was used for the T. reesei sample. The results of the catalase assay are shown in Table 2 (absorbance at 240 nm).

TABLE 2
Activity of A. niger Catalase Expressed in A. niger and T. reesei

| Time | Control | | |
|---|---|---|---|
| (minutes) | (no catalase) | A. niger | T. reesei |
| 0 | 1.011 | 0.977 | 0.968 |
| 1 | — | 0.552 | 0.632 |
| 2 | — | 0.385 | 0.462 |
| 3 | — | 0.276 | 0.356 |
| 4 | — | 0.203 | 0.282 |
| 5 | 1.013 | 0.156 | 0.234 |
| 6 | — | 0.119 | 0.199 |
| 7 | — | 0.095 | 0.0169 |
| 8 | — | 0.082 | 0.144 |
| 9 | — | 0.070 | 0.127 |
| 10 | 1.020 | 0.054 | 0.114 |
| 11 | — | — | — |
| 12 | — | 0.045 | 0.090 |
| 13 | — | — | — |
| 14 | — | 0.037 | 0.074 |
| 15 | 1.015 | — | — |
| 16 | — | 0.045 | 0.064 |
| 17 | — | — | — |
| 18 | — | 0.028 | 0.060 |
| 19 | — | — | — |
| 20 | 1.020 | 0.029 | 0.052 |

Evaluation of the Performance of Aspergillus niger Catalase Expressed in Aspergillus niger, Trichoderma reesei, and Trichoderma reesei with Endo-T Deletion

Catalase activity was determined for the samples shown in Table 3.

TABLE 3
Catalase Samples

| Sample | Activity (U/g) | Weight (g)/10 ml | Dose (ml) |
|---|---|---|---|
| A. niger | 135 750 | 0.1109 | 0.300 |
| T. reesei | 133 870 | 0.1199 | 0.304 (+8%) |
| T. reesei with | 20 000 | 0.1108 | 2.036 |
| Endo-T deletion | | | |

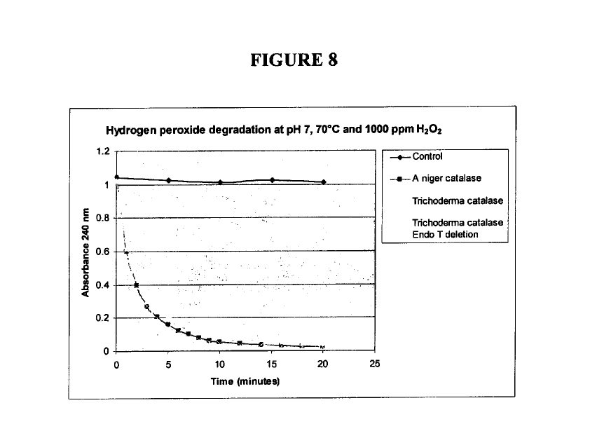

The results are shown in Table 4 (absorbance at 240 nm) and FIG. 8.

TABLE 4
Activity of A. niger Catalase Expressed in A. niger, T. reesei,
and T. reesei with Endo-T deletion

| | | | | T. reesei with |
|---|---|---|---|---|
| Time | Control | | | Endo-T |
| (minutes) | (no catalase) | A. niger | T. reesei | deletion |
| 0 | 1.044 | 1.044 | 1.026 | 1.025 |
| 1 | — | 0.602 | 0.618 | 0.607 |
| 2 | — | 0.394 | 0.437 | 0.382 |
| 3 | — | 0.268 | 0.325 | 0.270 |
| 4 | — | 0.205 | 0.265 | 0.199 |
| 5 | 1.026 | 0.156 | 0.212 | 0.141 |
| 6 | — | 0.124 | 0.173 | 0.108 |
| 7 | — | 0.099 | 0.142 | 0.082 |
| 8 | — | 0.080 | 0.122 | 0.065 |
| 9 | — | 0.063 | 0.103 | 0.051 |
| 10 | 1.013 | 0.053 | 0.095 | 0.043 |
| 11 | — | — | — | — |
| 12 | — | 0.042 | 0.075 | 0.034 |
| 13 | — | — | — | — |
| 14 | — | 0.036 | 0.065 | 0.036 |
| 15 | | — | — | — |
| 16 | — | 0.032 | 0.052 | 0.028 |
| 17 | — | — | — | — |
| 18 | — | 0.026 | 0.045 | 0.022 |
| 19 | — | — | — | — |
| 20 | 1.013 | 0.020 | 0.042 | 0.020 |

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for producing a polypeptide capable of catalyzing enzymatic conversion of hydrogen peroxide to oxygen and water, comprising expressing said polypeptide from a polynucleotide that encodes the polypeptide in a Trichoderma host cell.

2. A method according to claim 1, wherein said polypeptide is a catalase enzyme from Aspergillus niger (A. niger).

3. A method according to claim 1, wherein said polynucleotide comprises the A. niger catR gene.

4. A method according to claim 1, wherein said polynucleotide encodes a polypeptide comprising the amino acid sequence set forth in SEQ ID NO:1.

5. A method according to claim 4, wherein said Trichoderma host cell is a Trichoderma reesei (T. reesei) cell.

6. A method according to claim 5, wherein expression of said polypeptide in T. reesei is at least about 50% greater than expression of the same polypeptide in A. niger.

7. A method according to claim 5, wherein the amount of said polypeptide secreted into the growth medium from said T. reesei host cell is at least about 80% higher than the amount of said polypeptide secreted into the growth medium from an A. niger host cell.

8. A method according to claim 1, wherein the Trichoderma host cell comprises a deletion of the endo-T gene.

9. A method for producing a polypeptide capable of catalyzing enzymatic conversion of hydrogen peroxide to oxygen and water, comprising:
(a) transforming a Trichoderma host cell with an expression vector comprising a polynucleotide that encodes said polypeptide;
(b) growing said Trichoderma host cell in a growth medium under conditions suitable for expression of said polypeptide; and
(c) isolating said polypeptide from said growth medium.

10. A method according to claim 9, wherein said polypeptide is a catalase enzyme from A. niger.

11. A method according to claim 9, wherein said polynucleotide comprises the A. niger catR gene.

12. A method according to claim 9, wherein said polynucleotide encodes a polypeptide comprising the amino acid sequence set forth in SEQ ID NO:1.

13. A method according to claim 12, wherein said Trichoderma host cell is a T. reesei cell.

14. A method according to claim 13, wherein expression of said polypeptide in T. reesei is at least about 50% greater than expression of the same polypeptide in A. niger.

15. A method according to claim 14, wherein the amount of said polypeptide secreted into the growth medium from said T. reesei host cell is at least about 80% greater than the amount of said polypeptide secreted into the growth medium from an A. niger host cell.

16. A method according to claim 9, wherein the Trichoderma host cell comprises a deletion of the endo-T gene.

17. A polypeptide that is capable of catalyzing enzymatic conversion of hydrogen peroxide to oxygen and water, wherein said polypeptide is produced according to the method of claim 1.

18. A polypeptide according to claim 17, wherein the Trichoderma host cell comprises a deletion of the endo-T gene.

19. A polypeptide that is capable of catalyzing enzymatic conversion of hydrogen peroxide to oxygen and water, wherein said polypeptide is produced according to the method of claim 9.

20. A polypeptide according to claim 19, wherein the Trichoderma host cell comprises a deletion of the endo-T gene.

21. A method for converting hydrogen peroxide to oxygen and water, comprising contacting said hydrogen peroxide with the polypeptide of claim 17.

22. A composition for conversion of hydrogen peroxide to oxygen and water, comprising the polypeptide of claim 19.
